(19) United States
(12) Patent Application Publication
(10) Pub. No.: US 2009/0253895 A1
(43) Pub. Date: Oct. 8, 2009

(54) Printable Materials

(75) Inventors: Yoav Eichen, Haifa (IL); Nir Tessler, Zikhron-Yaakov (IL); Olga Solomeshch, Haifa (IL); Batia Blumer Gonen, Kiryat-Ata (IL)

Correspondence Address: MARTIN D. MOYNIHAN d/b/a PRTSI, INC., P.O. BOX 16446, ARLINGTON, VA, 22215, US

(73) Assignee: Technion Research & Development Foundation Ltd., Haifa (IL)

(21) Appl. No.: 11/990,437

(22) PCT Filed: Aug. 15, 2006
(86) PCT No.: PCT/IL06/00945

§ 371 Date: Feb. 14, 2008

Related U.S. Application Data
(60) Provisional application No. 60/707,967, filed on Aug. 15, 2005.

(51) Int. Cl. C07K 9/00; C07K 1/00; C07K 5/06; C07K 14/00; C07K 7/08; C07K 7/06; C07K 5/10; C07K 5/08

(52) U.S. Cl. 530/322; 530/300; 530/324; 530/325; 530/326; 530/327; 530/328; 530/329; 530/330; 530/331

(57) ABSTRACT

The present invention provides compounds of the general formula I, and uses thereof for printing electronic components such as wires, resistors and LEDs.

### FIELD OF THE INVENTION

This invention relates to compounds for use as printable materials.

### BACKGROUND OF THE INVENTION

Organic polymers and molecules are becoming major players in low cost electronics and optoelectronics. At present, solution processed polymers have an inherent advantage since a solution containing these polymers may be used for printing electronic components such as wires, resistors and emissive layers of light emitting diodes. However, to arrive at high quality printing it is not sufficient to merely make a solution of these polymers, as it is essential also to match the solution properties e.g. viscosity, to the printing technique to be used (see D. MacKenzie, Tutorial, MRS 2005).

Standard methods used in conventional printing do not apply to solution processed polymers, as with this method it is usually undesirable to mix, blend or dilute the active materials, i.e, the polymers, in inert materials since such processing will not only affect the solution properties but also modify the electronic properties of the printed layer, and thus possibly make the mixture useless.

The alternative method that has been developed (S. Shaked, S. Tal, Y. Roichman, A. Razin, S. Xiao, Y. Eichen, and N. Tessler, “Charge density and film morphology dependence of charge mobility in polymer field-effect transistors,” Advanced Materials, vol. 15, pp. 913, 2003) is to fine-tune the molecular weight of the organic polymers. This method, however, has two disadvantages: 1. the tuning is not trivial and only a small viscosity range is typically achieved; and 2. the physical arrangement (morphology) of the polymer is linked to its electronic properties and hence changing the viscosity by increasing the molecular weight will hinder previously optimized electronic properties.

Thus, there is has been an industrial need for the production of an organic-polymer based printing material which would have the solution and film forming properties which are necessary in order to achieve a film of the required viscosity, adhesion to the surface and uniformity with minimal domain boundaries that would render to it the desired electronic and/or optoelectronic properties. In the absence of such properties the polymer would be considered not useful as a printing material for the manufacture or printing of, for example, light emitting diodes (display & lighting type applications), printing of electronic circuits as field effect transistors, capacitors, and diodes for low cost logic, smart barcodes/tags, RFID, solar cells or other light detectors, sensors for chemical and/or biological moieties and also for printing of labels or indicators with unique signatures.

### SUMMARY OF THE INVENTION

It has now been surprisingly found that solution and film properties of various polymers may be finely tuned by constructing polymers (e.g. peptides, peptide nucleic acids (PNA) and nucleic acids) with so-called “solution-modifying units” and/or with “film-forming units” which impart to these polymers the required electronic and photoelectronic properties. Such polymers minimize or diminish the need for formulation additives to control the solution and film properties of the printable material.

The construction of such polymers was achieved by employing various synthetic methods, one of which being the use of tailor-made monomeric building blocks, each having the capability of imparting to the constructed oligomer or polymer at least one property selected from solubility, viscosity, film-forming, adhesivity, electronic, photoelectronic and magnetic.

Thus, in a first aspect of the present invention, there is provided a monomeric building block of the general formula I:

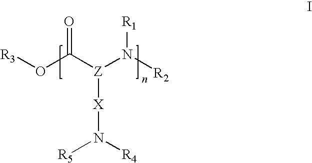

wherein

R1 and R2, independently of each other, are selected from H, C1-C20 alkyl, C2-C20 alkenyl, C2-C20 alkynyl, C1-C20 alkylene, C2-C20, alkenylene, C2-C20 alkynylene, silyl, C1-C20 alkylene carbonyl nucleobase, and N-protecting group;

R3 is selected from H and an O-protecting group;

R4 and R5, independently of each other, are selected from H, C1-C20 alkyl, C2-C20 alkenyl, C2-C20 alkynyl, C1-C20 alkylene, C2-C20 alkenylene, C2-C20 alkynylene, cycloalkyl, cycloalkenyl, cycloalkynyl, aryl, arylene, heteroaryl, heteroarylene, aralkyl, heteroaralkyl, haloalkyl, alkoxy, haloalkoxy, sulfonyl, carboxy, alkylaminocarbonyl, and a radical of the general formula II:

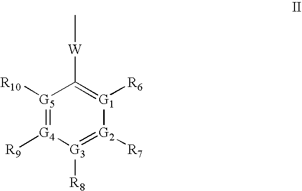

wherein each of R6 to R10, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl;

two vicinal (i.e., neighboring) groups of R6 to R10 may together with the carbon atoms to which they are bonded form a substituted or unsubstituted C5-C10 fused ring system selected from cycloalkyl, cycloalkenyl, cycloalkynyl, heterocyclic, and arylene; said fused ring system may contain at least one heteroatom selected from O, N or S;

each of G1 to G5 may be an atom selected from C, O, N or S; where the atom G1, G2, G3, G4 or G5 is different from C, the atom may be charged or neutral; when atom G1 to G5 is different from C, the atom may or may not be substituted as shown; in case of substitution, said atom (G1 to G5) may be positively charged; when charged, the system may be accompanied by a counter ion selected from negatively inorganic or organic anions;

W is a group selected from —C(O)—, —S(O)— and —S(O)₂—;

R4 and R5 together with the N atom to which they are bonded, may form a heterocyclic ring structure having optionally at least one additional heteroatom selected from N, O or S; said ring structure being selected from substituted or unsubstituted pyridine, isoquinoline, benzoisoquinoline, benzoisoquinoline-1-one, isobenzoquinoline-1,3-dione, benzo[1,7]naphthyridine dione, and 1,6,8-triazaphenalen-7,9-dione;

Z is selected from C1-C2 alkylene, C5-C8 cycloalkylene, C5-C10 arylene, C5-C12 heteroarylene having at least one heteroatom selected from N, O, and S;

X is selected from C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl; and

n is an integer being equal or greater than 1; wherein when n is greater than 2, R1 or R2 is a peptide bond.

In one embodiment, in the general formula I, Z is —CH—, X is C1-C20 alkylene, R4 is H and R5 is selected from C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, alkylaminocarbonyl, or a radical of the general formula IIa:

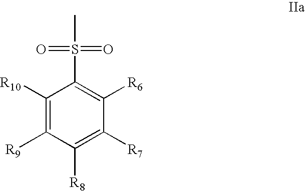

wherein two vicinal groups of R6 to R10 may together with the carbon atoms to which they are bonded form a C5-C10 fused ring system selected from cycloalkyl, cycloalkenyl, cycloalkynyl, heterocyclic, and arylene; said fused ring system optionally containing at least one heteroatom selected from N, O and S, and wherein each of R6 to R10 is as defined hereinabove.

In another embodiment, in the general formula I, X is C1-C4 alkylene, R6, R7, R9 and R10 are each H and R8 is selected from C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

In yet another embodiment, the compound of the general formula I is of the general formula III:

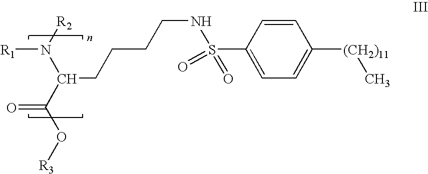

wherein each of R1 to R3 and n are as defined hereinbefore.

In a particular embodiment of the present invention, there is provided a compound of the general formula III, wherein R1, R2, and R3 are each H, and n is 1, said compound herein designated as Compound A:

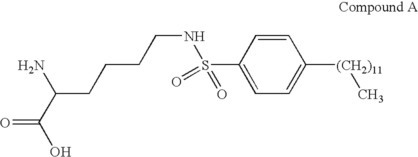

In another particular embodiment, there is provided a compound of the general formula III, wherein R1 is H, R2 is a peptide bond and n=2, said compound herein designated Compound A2,

or when n=3, said compound herein designated Compound A3,

or when n=4, said compound herein designated Compound A4,

or when n=5, said compound herein designated Compound A5,

or when n=6, said compound herein designated Compound A6,

or when n=7, said compound herein designated Compound A7,

or when n=8, said compound herein designated Compound A8,

or when n=9, said compound herein designated Compound A9,

or when n=10, said compound herein designated Compound A10,

or when n is greater than 10, the compounds are designated as Compound A11, A12, A13, etc.

In yet another embodiment of the present invention, in the general formula I, Z is a —CH—, X is C1-C20 alkylene, R4 is H and R5 is a radical of the general formula IIa, wherein R6, R7 and R8 are as defined hereinabove, and R9 and R10 together with the carbon atoms to which they are bonded form a C5-C10 fused ring system selected from cycloalkyl, cycloalkenyl, cycloalkynyl, heterocyclic, and arylene.

In a particular embodiment, in the compound of general formula I, said C5-C10 fused ring system is a substituted or unsubstituted naphthalenyl, said compound is of the general formula IV:

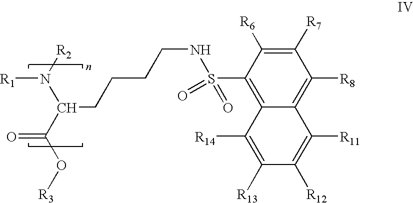

wherein each of n, R1 to R3 and R6 to R8 is as defined hereinabove, each of R11 to R14, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

Preferably, R6, R7, R8, R12, R13, and R14 are each H and R11 is —NRR′, wherein R and R′ may be identical or different and may, independently of each other, be selected from H, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl. Each of said R and R′, independently of each other may be further substituted.

R and R′ may also together with the N atom to which they are bonded, form a heterocyclic ring structure selected from substituted or unsubstituted heterostructures, e.g. pyridine, isoquinoline, benzoisoquinoline, benzoisoquinoline-1-one, isobenzoquinoline-1,3-dione, benzo[1,7]naphthyridine dione, 1,6,8-triazaphenalen-7,9-dione and derivatives thereof.

In a particular embodiment, the compound of the general formula IV is of the general formula V:

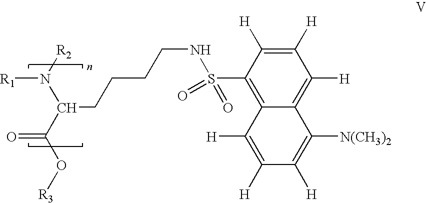

wherein, n and R1 to R3 are as defined hereinabove.

In a particular embodiment of the present invention, there is provided a compound of the general formula V, wherein R1, R2, and R3 are each H, and n is 1, said compound herein designated as Compound B:

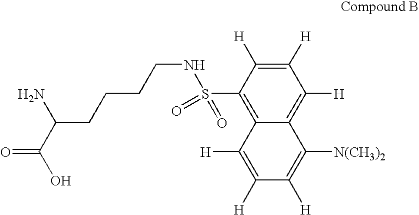

In another particular embodiment, there is provided a compound of the general formula V, wherein R1 is H, R2 is a peptide bond and n=2, said compound herein designated Compound B2,

or when n=3, said compound herein designated Compound B3,

or when n=4, said compound herein designated Compound B4,

or when n=5, said compound herein designated Compound B5,

or when n=6, said compound herein designated Compound B6,

or when n=7, said compound herein designated Compound B7,

or when n=8, said compound herein designated Compound B8,

or when n=9, said compound herein designated Compound B9,

or when n=10, said compound herein designated Compound B10,

or when n is greater than 10, the compounds are designated as Compound B11, B12, B13, etc.

In another embodiment of the present invention, in the general formula I, Z is —CH—, X is C4 alkylene, and R4 and R5 together with the N atom to which they are bonded, form a heterocyclic ring structure selected from substituted or unsubstituted pyridine, isoquinoline, benzoisoquinoline, benzoisoquinoline-1-one, isobenzoquinoline-1,3-dione, benzo[1,7] naphthyridine dione, and 1,6,8-triazaphenalen-7,9-dione. Preferably, R4 and R5 together with the N atom to which they are bonded, form a heterocyclic ring structure selected from substituted or unsubstituted benzoisoquinoline, benzoisoquinoline-1-one, isobenzoquinoline-1,3-dione and derivatives thereof.

In a particular embodiment of the present invention, R4 and R5 together with the N atom to which they are bonded form an isobenzoquinoline-1,3-dione ring structure, as shown in the general formula VI:

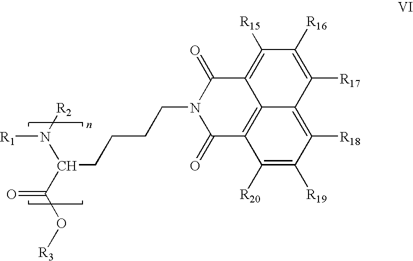

wherein each of n and R1 to R3 is as defined hereinabove and each of R15 to R20, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

In a further embodiment, in the general formula VI, each of said R15 to R20, independently of each other, is selected from H, hydroxyl, C1-C20 alkoxy, and substituted or unsubstituted amine. Preferably, said amine is —NR21R22, wherein said R21 and R22, independently of each other is H or a C1-C20 alkyl group; more preferably R21 and R22 are each C1-C20 alkyl group and most preferably said amine is situated at either or both R17 or R18.

In another embodiment, in the compound of the general formula VI, R17 is substituted by NR21R22, as defined hereinabove and R18 is H, said compound is of the general formula VII:

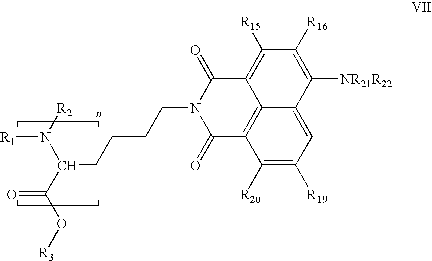

and wherein each of n, R1 to R3 and R15, R16, R19 and R20 is as defined herein.

In yet another embodiment, in the general formula VII, each of R15, R16, R19 and R20, independently of each other is H, hydroxyl, alkoxy or aryloxy and R21 and R22 is a C1-C20 alkyl group.

In a particular embodiment, in the general formula VII, R21 is a methyl or an ethyl and R22 is selected from C1-C8 alkyl (e.g. methyl, ethyl, propyl, isopropyl, butyl, t-butyl, pentyl, hexyl, heptyl, 3-octyl, 2-octyl, and octyl), being optionally straight or branched or optionally further substituted, and R15, R16, R19 and R20 may each be H, hydroxyl, alkoxy or aryloxy in one of the following combinations:
1. each of R15, R16, R19 and R20 is H;
2. R15 and R20 are each selected from hydroxyl, alkoxy and aryloxy and R16 and R19 are H;
3. R16 and R19 are each selected from hydroxyl, alkoxy and aryloxy and R15 and R20 are H;
4. R16 and R20 are each selected from hydroxyl, alkoxy and aryloxy and R15 and R19 are H;
5. R15 and R19 are each selected from hydroxyl, alkoxy and aryloxy and R16 and R20 are H;
6. R15 and R16 are each selected from hydroxyl, alkoxy and aryloxy and R19 and R20 are H;
7. R19 and R20 are each selected from hydroxyl, alkoxy and aryloxy and R15 and R16 are H; or
8. each of R15, R16, R19 and R20 is selected from hydroxyl, alkoxy and aryloxy.

In a particular embodiment, the compound of the general formula VII is the compound of the general formula VIII:

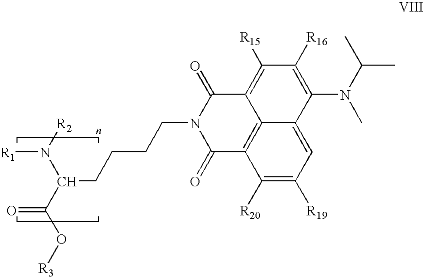

wherein each of n, R1 to R3 and R15, R16, R20 and R19 are as defined hereinabove.

In another particular embodiment, the compound of the general formula VII is the compound of the general formula IX:

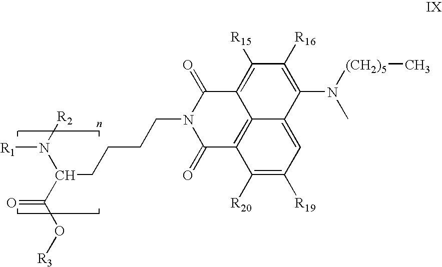

wherein each of n, R1 to R3 and R15, R16, R20 and R19 are as defined hereinabove.

In another particular embodiment, the compound of the general formula VII is the compound of the general formula X:

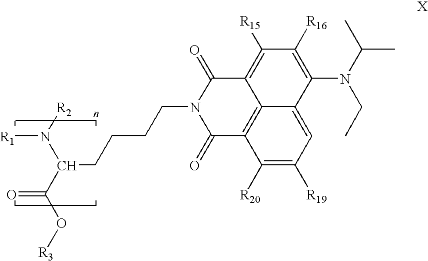

wherein each of n, R1 to R3 and R15, R16, R20 and R19 are as defined hereinabove.

In another particular embodiment, the compound of the general formula VII is the compound of the general formula XI:

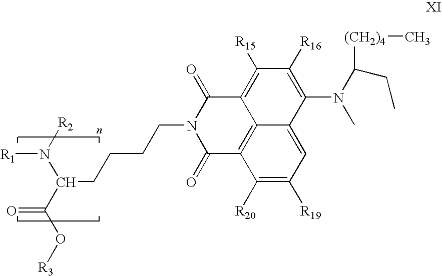

wherein each of n, R1 to R3 and R15, R16, R20 and R19 are as defined hereinabove.

For each compound of the general formulas VIII to XI, each of R15, R16, R19 and R20 may be, independently of each other H, hydroxyl, alkoxy or aryloxy in one of the following combinations:
1. each of R15, R16, R19 and R20 is H;
2. R15 and R20 are each selected from hydroxyl, alkoxy and aryloxy and R16 and R19 are H;
3. R16 and R19 are each selected from hydroxyl, alkoxy and aryloxy and R15 and R20 are H;
4. R16 and R20 are each selected from hydroxyl, alkoxy and aryloxy and R15 and R19 are H;
5. R15 and R19 are each selected from hydroxyl, alkoxy and aryloxy and R16 and R20 are H;
6. R15 and R16 are each selected from hydroxyl, alkoxy and aryloxy and R19 and R20 are H;
7. R19 and R20 are each selected from hydroxyl, alkoxy and aryloxy and R15 and R16 are H; or
8. each of R15, R16, R19 and R20 is selected from hydroxyl, alkoxy and aryloxy.

In another particular embodiment, there is provided a compound of the general formula VIII, or XI, or X or XI, wherein R1 is H, R2 is a peptide bond and n=2, or n=3, or n=4, or n=5, or n=6, or n=7, or n=8, or n=9, or n=10, or n is greater than 10, etc.

In another embodiment, in the general formula I, Z is a C5-C10 arylene or a C5-C12 heteroarylene having at least one heteroatom selected from N, O and S. In a preferred embodiment, said C5-C10 arylene is selected from substituted or unsubstituted phenyl and naphthyl and said C5-C12 heteroarylene is selected from thiophenyl, thiozylyl, and imidazolyl.

In yet another embodiment of the compound of general formula I, Z is —CH—, X is C1-C20 alkylene, R4 is H and R5 is selected from H, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, alkylaminocarbonyl, or a radical of the general formula IIb:

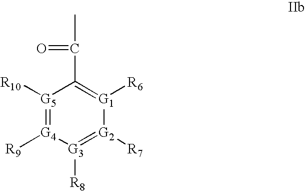

wherein each of R6 to R10, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl;

each of G1 to G5 may be an atom selected from C, O, N or S; where the atom G1, G2, G3, G4 or G5 is different from C, the atom may be charged or neutral; when atom G1 to G5 is different from C, the atom may or may not be substituted as shown; in case of substitution, said atom (G1 to G5) may be positively charged; when charged, the system may be accompanied by a counter ion selected from negatively inorganic or organic anions;

two vicinal (i.e., neighboring) groups of R6 to R10 may together with the carbon atoms to which they are bonded form a substituted or unsubstituted C5-C10 fused ring system selected from cycloalkyl, cycloalkenyl, cycloalkynyl, heterocyclic, and arylene.

In a particular embodiment, R4 is H and R5 is a radical of the general formula IIb, wherein one or two of said G1 to G5 atoms are heteroatoms selected from N and O.

In yet another embodiment, the compound of the general formula I is of the general formula XII:

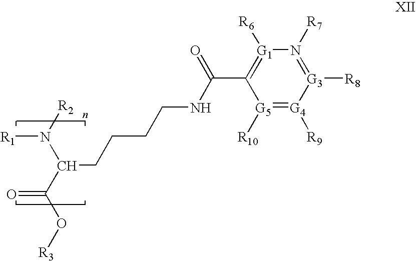

wherein each of n, R1 to R3, R6 and R8 to R10 is as defined hereinabove, and R7 may be absent. In one case, R7 is absent and thus the N atom is uncharged. In another case, R7 is present and the N atom is positively charged. R7 is as defined above.

In another embodiment, in general formula XII, R6, R9 and R10 are H, R7 is absent and R8 is a heteroaryl selected from substituted or unsubstituted pyridyl, thiophenyl, isoquinolinyl, benzoisoquinolinyl, and derivatives thereof. Preferably, the heteroaryl is a substituted pyridyl. In a more preferred embodiment, the pyridyl is 2-pyridyl. In a most preferred embodiment, the compound of the general formula I is of the general structure XIII:

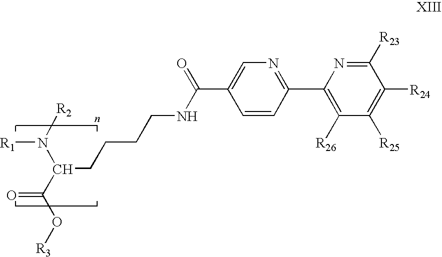

wherein each of n, R1 through R3 are as defined hereinabove and wherein each of R23 to R26, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

In a preferred embodiment, in the general formula XIII, at least one of said R23 to R26 is a conjugated C2-C20 alkenylene, C2-C20 alkynylene, arylene or heteroarylene or a combination thereof.

In a more preferred embodiment, R23, R25 and R26 are H and R24 is a conjugated C2-C20 alkenylene, C2-C20 alkynylene, arylene or heteroarylene or a combination thereof.

In a particular embodiment, a compound of the general formula XIII is a compound of the general structure XIV:

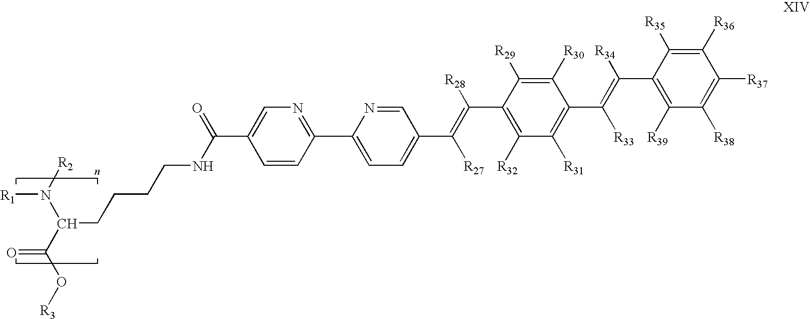

wherein each of n and R1 through R3 is as defined hereinabove and wherein each of R27 to R39, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

In one embodiment, in the compound of general formula XIV, R27, R28, R33 and R34 are H. The compound of the general formula XIV may be all-cis or all-trans or one bond in the cis and the other in the trans configuration.

In another embodiment, in the general formula XIV, R37 may be H or a conjugated C2-C20 alkenylene, C2-C20 alkynylene, arylene or heteroarylene or a combination thereof.

In still another embodiment, in the general structure XIV, R30, R32, R36 and R39 are each, independently selected from a substituent other than H and R29, R31, R35 and R38 are each H. Preferably, said substituent other than H is selected from hydroxyl, alkoxy, and aryloxy, thus providing a structure of the general formula XV:

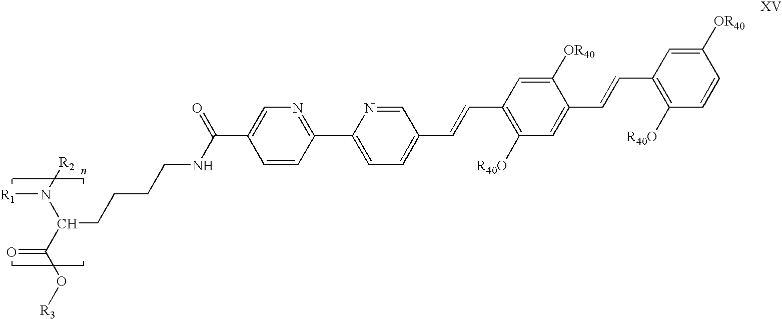

wherein each of n and R1 to R3 are as defined hereinabove and R40 is selected from H, C1-C20 alkylene, and C6-C15 arylene.

In another aspect of the present invention, there is provided a compound of the general formula I, as well as of any compound of the general formulas III through XV, wherein n is greater than 1. These compounds include each at least one peptide bond connecting between any two amino acid moieties.

These peptides or oligomers may be constructed as homo-oligomers or homopolymers, namely of identical compounds of the general formulas of the invention or different compounds of the general formulas of the invention.

In one embodiment, the oligomer comprises identical monomers of the general formula I connected to its neighboring monomer via a peptide bond.

In another embodiment, the oligomer comprises monomers of different structures.

As known to a person skilled in the art, a dimer of two different amino acids, e.g. Compound A and Compound B of general formula I may afford two different structures: A-B and B-A, which are different from each other. Both generalized structures are encompassed in the scope of the present invention.

Thus, the invention also provides any compound, being a monomer, an oligomer, or a polymer of the general formula I having between 1 and 100 repeating or in a random combination of the monomers.

In one embodiment, there is provided the oligomer constructed of repeating monomers of Compounds A and B and having the general structure BABABA, wherein the number of monomers of Compound A equals the number of monomers of Compound B. This oligomer (n=6), designated herein as Compound C is of the following structure:

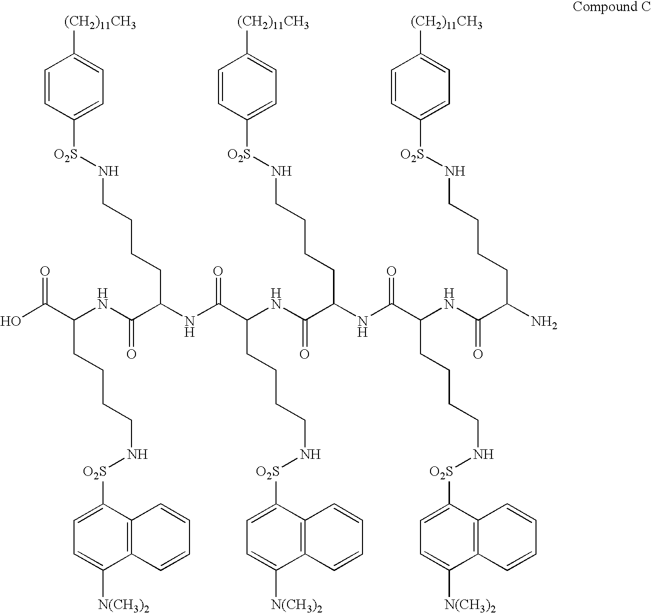

The N-terminal of Compound C, or the C-terminal thereof, or of any other compound of the present invention, may be substituted with a terminating group such as an N- or O-protecting group or other non-reactive group. Such terminating group may be for example a long chain alkanoic acid such as 2-hexyl-1-undecanoic acid or any other terminating group. The terminating group is herein designated by the latter T.

In another embodiment, there is provided an oligomer having a construction of four monomers of Compound B to only two monomers of Compound A. This oligomer (n=6) is designated as Compound D:

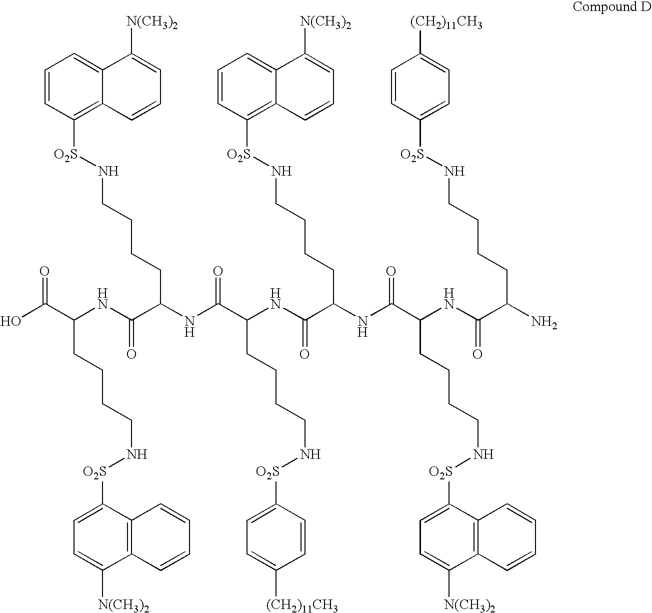

Compound D as well may have T groups at either or both of its terminals.

In another embodiment, there is provided an oligomer having a construction of 10 monomers of the compound of the general formula IX, said oligomer herein designated Compound E:

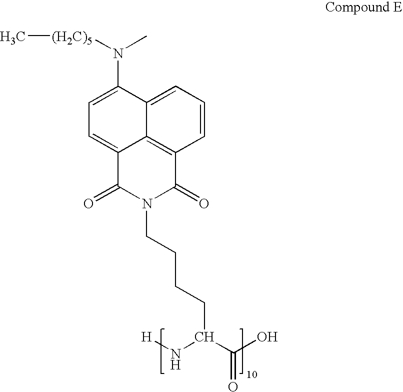

Compound E as well may have T groups at any one or both of its terminals.

In yet another aspect of the present invention, there are provided polymers, preferably homopolymers of the compounds of the present invention.

Each of said oligomers or polymers have film forming properties, and electronic or photoelectronic properties, as will be shown next.

The monomers, oligomers and polymers of the present invention may be used as means to control and tailor adhesion properties to specific surfaces. These compounds may also be used to provide thermally activated and/or photoinduced cross-linking capabilities such as catalysis.

The compounds of the invention, and particularly those having luminescent properties, i.e., the compounds of the general formulas VI through XI, may also be used for the preparation of materials that posses the desired luminescent properties together with optimized high quality printability.

The compounds of the present invention may also be used in the constructions of electronic materials and electronic components such as active layers in light emitting diodes, diodes, resistors, capacitors, transistors and sensors. The application of the specific materials is preferably either as insulators or organic semiconductors or as conductors in the aforementioned devices.

The compounds may also be used as sensing components for sensing the presence of a certain analyte (an agent is the gas, liquid or solid state, including in mixtures), in response to which presence they change at least one of their electronic or photoelectronic properties (such as photoluminescence, capacitance, resistance) or a change in said property as a result of e.g., analyte interaction therewith.

### BRIEF DESCRIPTION OF THE DRAWINGS

In order to understand the invention and to see how it may be carried out in practice, a preferred embodiment will now be described, by way of non-limiting examples only, with reference to the accompanying drawings, in which:

FIG. 1: Microscope images of films made of BABABA (left) and BBABBA (right). The upper two were taken with a magnification of ×50 and the lower two with a magnification of ×100. Films thicknesses were 80 nm and 60 nm for the BABABA and BBABBA, respectively.

FIG. 2: AFM images on a 1×1 μm range. The left pictures were taken for the BABABA film and the right pictures were taken for the BBABBA. Both the height and phase images look very similar in the two films. However, the scale for the phase image is three times larger in the BBABBA case.

FIG. 3: structurally similar luminescent amino acids for optimization of luminescence and printability properties. The R groups are as described in reference to general formulas VI to XI.

FIGS. 4A-K: Non-limiting examples of conjugated and non conjugated amino acids, wherein each of the arylenes and/or heteroarylenes are optionally substituted as disclosed in reference to general formula I.

FIG. 5: A scheme exemplifying the use of a compound of the present invention as a sensing molecule for the presence of an analyte.

FIGS. 6A-C: FIG. 6A shows the PL spectrum of the derivative of Compound E; FIG. 6B shows the structure of a LED composing the derivative of Compound E; and FIG. 6C shows the LED characteristics.

### DETAILED DESCRIPTION OF THE INVENTION

The oligomers or polymers of the invention, to which film forming properties have been imparted by chemical tailoring of monomeric structures, may comprise a backbone of various lengths. While the invention disclosed herein specifically exemplifies the use of polypeptides as the preferred backbone, it should be understood to a person skilled in the art that similar chemical tailoring can also afford polymers of nucleic acids or peptide nucleic acids (PNA) having the required properties.

As detailed hereinabove, the first aspect of the present invention provides monomeric residues which may be bonded to each other, by any method known to a person skilled in the art, to form dimers, trimers, quartermers, or longer oligomers or polymers to suit the requirements of the specific application. The backbone of such oligiomers or polymers is preferably peptidic in nature and made of repeating residues of conjugated or non-conjugated amino acids of the general formula I, wherein each of the groups is as defined hereinbefore.

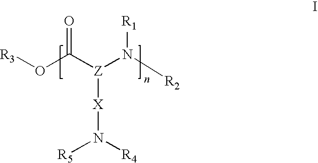

It is to be understood that the compounds of the present invention, namely the monomeric building blocks as well as the oligomers and polymers may contain chiral centers. Such chiral centers may be of either the (R) or (S) configuration, or may be a mixture thereof. Thus, the compounds provided herein may be enantiomerically pure, or be stereoisomeric or diastereomeric mixtures.

The term “alkyl”, if not specified, refers to a carbon chain having from 1 to 20 carbon atoms, being straight or branched, and may or may not be substituted.

The term “alkenyl” refers to a carbon chain of from 2 to 20 carbons and containing 1 to 8 double bonds, being straight or branched and may or may not be substituted. Each of said double bonds may be in the cis or trans configuration.

The term “alkynyl” refers to a carbon chain of 2 to 20 carbons, containing 1 to 8 triple bonds and being straight or branched and optionally substituted.

Exemplary alkyl, alkenyl and alkynyl groups herein include, but are not limited to, methyl, ethyl, propyl, isopropyl, isobutyl, n-butyl, sec-butyl, tert-butyl, isohexyl, allyl (propenyl) and propargyl (propynyl).

As used herein, “alkylene” refers to a straight, branched or cyclic, in certain embodiments straight or branched, aliphatic hydrocarbon group, in one embodiment having from 1 to about 20 carbon atoms, in another embodiment having from 1 to 12 carbons. There may be optionally inserted along the alkylene group one or more oxygen, sulfur, including S(═O) and S(═O)₂ groups, or substituted or unsubstituted nitrogen atoms including —NK— and —N⁺KK— groups, where the nitrogen substituent(s), K, is(are) alkyl, aryl, aralkyl, heteroaryl, heteroaralkyl or COK′, where K′ is alkyl, aryl, aralkyl, heteroaryl, heteroaralkyl, —OY or —NYY, where Y is hydrogen, alkyl, aryl, heteroaryl, cycloalkyl or heterocyclyl.

Alkylene groups include, but are not limited to, methylene (—CH₂), ethylene (—CH₂CH₂—), propylene (—(CH₂)₃—), methylenedioxy (—O—CH₂—O—) and ethylenedioxy (—O—(CH₂)₂—O—).

As used herein, “alkylene carbonyl nucleobase” refers to alkylene-CO-base, wherein the alkylene is as defined herein and the nucleobase is selected from purines and pyrimidines, e.g., adenine, guanine, thymine, cytosine and uracil.

As used herein, “alkenylene” refers to a straight, branched or cyclic, in one embodiment straight or branched, aliphatic hydrocarbon group, in certain embodiments having from 2 to about 20 carbon atoms and at least one double bond, in other embodiments 1 to 12 carbons. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂.

The term “alkynylene” refers to a straight, branched or cyclic, in certain embodiments straight or branched, aliphatic hydrocarbon group, in one embodiment having from 2 to about 20 carbon atoms and at least one triple bond, in another embodiment 1 to 12 carbons. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multi-cyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or sprio-connected fashion.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

The term “arylene” refers to a monocyclic or polycyclic, in certain embodiments monocyclic, aromatic group, in one embodiment having from 5 to about 20 carbon atoms and at least one aromatic ring, in another embodiment 5 to 12 carbons. Arylene groups include, but are not limited to, 1,2-, 1,3- and 1,4-phenylene.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, N, O or S. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heteroarylene” refers to a monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, N, O or S.

As used herein, “aralkyl” refers to an alkyl group in which one of the hydrogen atoms of the alkyl is replaced by an aryl group, as defined herein.

As used herein, “heteroaralkyl” refers to an alkyl group in which one of the hydrogen atoms of the alkyl is replaced by a heteroaryl group, as defined herein.

As used herein, “halo” or “halogen” refers to F, Cl, Br or I.

As used herein, “haloalkyl” refers to an alkyl group in which one or more of the hydrogen atoms are replaced by halogen, as defined. Such groups include, but are not limited to, chloromethyl, trifluoromethyl and 1-chloro-2-fluoroethyl.

As used herein, “alkoxy” and “alkylthio” refers to RO— and RS—, in which R is alkyl, as defined.

The term “aryloxy” refers to aryl-O— or -arylene-O—, wherein said aryl and arylene are as defined herein.

As used herein, “haloalkoxy” refers to RO— in which R is a haloalkyl group.

As used herein, “silyl” refers to —SiRRR, or —O—SiRRR, wherein R is an alkyl or an aryl as defined herein.

As used herein, “sulfinyl” or “thionyl” refers to —S(O)—.

As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “alkylaminocarbonyl” refers to —C(O)NHR and —C(O)NR′R in which R′ and R are each independently alkyl.

“Hydroxy” refers to —OH.

“Amine” refers to —NKK′ wherein each of K and K′ independently of each other, is selected from H and alkyl. The term also refers to charged ammonium groups.

As used herein, “amide” refers to the group —C(O)NH— or to —C(O)NRR′ in which each one of R and R′ is selected from H, alkyl and aryl.

The term “nitro” refers to —NO₂.

Each of the groups defined herein, where appropriate may be substituted with one or more substituents, in certain embodiments one, two, three or four substituents, where the substituents are any of the groups as defined herein.

Where the number of any given substituent is not specified (e.g., haloalkyl), there may be one or more substituents present. For example, “haloalkyl” may include one or more of the same or different halogens. As another example, “C₁₋₃alkoxyphenyl” may include one or more of the same or different alkoxy groups containing one, two or three carbons.

The term “oligomer”, as used herein, refers to a compound consisting of between 2 and 10 monomers (residues) of the invention which are chemically bonded to each other. The monomers may be different or same and may be arranged in a repetitive fashion such as in the case of BABABA, wherein the repeating unit is -(BA)- or a random fashion, wherein each monomer is a different compound of the invention. The oligomer may be constructed partially in a repetitive fashion and partially in a random fashion. The resulting oligomer may be a straight chain oligomer, having an overall a linear arrangement or may be substituted or branched. The term “oligo” wherever used herein, e.g. in oligopeptides, refers to a chain having between 2 and 6 units.

The term “polymer” refers within the context of the present invention to a compound consisting of 11 or more monomers which are bonded to one another. The monomers may be different or same and may be arranged as discussed herein. The resulting polymer may be a straight chain polymer, namely, having an overall a linear arrangement or may be substituted or branched. The term “poly” wherever used herein, e.g. in polypeptides, refers to a chain having at least 10 units. The term also encompasses homopolymers and copolymers constructed of the monomers of the invention.

Any one of the monomers, oligomers and polymers of the present invention may have partial or full substitution on the N atom of the amino acid monomers. The N atom may, for example be substituted by H and a peptide bond, or by an alkyl or silyl group and a peptide bond or may be fully substituted to afford a charged i.e., ammonium group. Thus, the monomers, oligomers and polymers of the invention may be neutral, charged or partially charged and may have any number of charged atoms. In case of positively charged systems, for example resulting from the presence of ammonium groups, the system may be accompanied by a counter ion selected from negatively inorganic or organic anions. Non-limiting examples of inorganic anions are Br⁻, Cl⁻, F⁻, I⁻, OH⁻, HS⁻, BrO₃⁻, BrO⁻, ClO₃⁻, ClO₄⁻, ClO₂⁻, ClO⁻, CrO₄²⁻, NO₃⁻, NO₂⁻, PO₄³⁻, HPO₄²⁻, H₂PO₄⁻, MnO₄⁻, SO₄²⁻, HSO₄⁻ and SO₃²⁻. Non-limiting examples of organic anions are CO₃², HCO₃⁻, HCO₂⁻, C₂O₄²⁻, HC₂O₄⁻, C₂H₃O₂⁻, OCN⁻, SCN⁻, and CN⁻. In case the compound is negatively charged, it may be accompanied by a positively charged counter ion, as known to a person skilled in the art.

The oligomers and polymers of the present invention may be synthesized by employing for example methodologies of peptide or nucleotide syntheses. Generally speaking, peptides are synthesized by chemically combining the carboxyl group of one amino acid with the amino group of another, forming a dimer or oligomer having a so-called C-terminus (carboxyl) and an N-terminus (amine).

Several methodologies are known for the synthesis of peptidic oligomers or polymers:

(1) Liquid-phase synthesis—This is one of the classical approaches to peptide synthesis which is mostly used in large-scale production of peptides for industrial purposes.

(2) Solid-phase synthesis—In this method, the amino acids are connected to each other step-by-step thus creating a pre-designed peptide or oligomer of a desired structure and molecular weight. This method allows the synthesis of peptides having a complex or unusual backbone modification and allows for the generation of high yield in each step. In a typical experiment, small beads or a different solid support is treated with linkers on which the peptidic oligomer chains may be built in a C-terminal to N-terminal fashion. In order to ensure complete coupling during each synthesis step, and avoid polymerization of the amino acids, each amino acid is presented semi-protected with a suitable N-terminal protecting group. Once the first amino acid has been bound to the support, the protective group is removed by deprotection, the deprotection reagents are washed away to provide clean coupling environment, the second and further protected amino acids (typically dissolved in a solvent such as dimethylformamide, DMF, combined with suitable coupling reagents) are presented to the synthesis medium and the process is repeated again (for further general information see C. A. Briehn and P. Bauerle, “Design and synthesis of a 256-membered, pi-conjugated oligomer library of regioregular head-to-tail coupled quater(3-arylthiophene)s,” J. Comb. Chem., vol. 4, pp. 457-469, 2002).

The oligomers and polymers of the present invention have been synthesized on a peptide synthesizer following this general methodology. For example, Compound C was prepared by first providing an Fmoc-protected Compound B, upon bond formation of protected Compound B to the support, the protection group was removed by exposure of the support to piperidine in DMF and a second molecule of Fmoc-protected Compound B was added. Deprotection was again performed and an Fmoc-procted Compound A was added. This procedure was followed until the support carried a protected form of Compound C. At this stage, the support was treated with TFA in order to afford Compound C. Alternatively, the protected oligomer was deprotected from the terminal Fmoc group and a terminating group T was added to afford a derivative of Compound C having the general structure BABABAT.

N-protecting groups are numerous and may be selected from carbobenzyloxy (Cbz) or benzyl (Bn) which may be removed by hydrogenolysis; t-butyloxycarbonyl (BOC) which may be removed by concentrated strong acid such as HCl or TFA; 9-fluorenylmethyloxycarbonyl (Fmoc) which may be removed by base, such as piperidine; and others known to a person skilled in the art. Preferably, the protecting groups are Fmoc or Boc.

(3) Fragment condensation—In this method, peptide fragments or short oligomers are coupled. Fragment condensation is better than stepwise elongation via the solid support for synthesizing sophisticated long peptides, but its use is restricted in order to protect against racemization. Fragment condensation is also undesirable since the coupled fragment must be in gross excess, which may be a limitation depending on the length of the fragment.

At times, it is necessary to protect the oxygen atom of the carboxyl end of the amino acid compounds of the invention. The O-protecting groups are also numerous and may be selected from various, such as methyl, benzyl, t-butyl, silyl and others, as known to a person skilled in the art (and as may for example be found in “Protective Groups in Organic Synthesis” by T. W. Greene and P. G. M. Wuts, 1999).

The building block monomers used in the construction of the oligomers or polymers of the invention allow the tailoring of compounds having film forming properties which are prerequisites for the formation of a continuous, flat top surface. Generally speaking, the oligomers and polymers of the invention are substituted with at least one solution-modifying monomer and/or at least one film-forming monomer.

The solution-modifying monomers are those capable of affecting the viscosity of the compound (e.g., oligomer or polymer) to which they are bonded. In one case, the viscosity of the compound will increase with an increase in the number of such monomers. In another case, the viscosity of the compound will decrease with an increase in the number of such substituting monomers.

The film-forming monomers are monomers which affect the wettability of the surface on which the compound bearing these groups is applied.

The combination of solution modifying and film forming monomers provides compounds which on one hand form films characterized by having: adhesiveness to various surfaces, low degree of crystallinity (namely, the film being preferably fully amorphous), minimum domain boundaries and flat top surface (less then 1% fluctuations in thickness), and on the other hand maintain the electronic and optoelectronic properties of the polymer.

The monomers of the invention may be constructed and arranged in the oligomer or polymer in any desirable arrangement in the presence or absence of any other residues capable of imparting other or additional opto- or electronic characteristics.

Compounds A and B of the invention were reacted with one another using an automated peptide synthesizer, forming two unique and novel sequences: BABABA, herein designated Compound C, and BBABBA, herein designated as Compound D. Each of these oligomers was terminated with a terminating long branched alkyl carboxylic acid group labeled T.

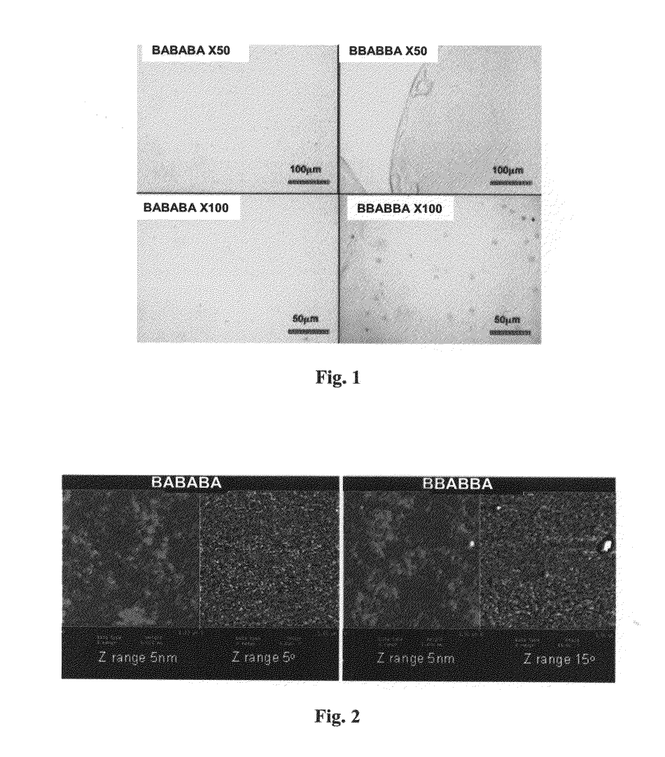

The solid and pure materials that were cleaved from the solid support of the peptide synthesizer were dissolved in anhydrous THF (10-20 mg/l ml) and spin-coated on a substrate such as glass resulting typically in films being 60-80 nm thick. FIG. 1 shows microscope images of the two films using magnification of ×50 and ×100. Films of the BABABA oligomer, shown on the left side, were highly uniform with a film boundary which circumferences the whole of the film. The small dots shown in the image result from an artifact found in the bare glass support. For the BBABBA oligomer, however, a film of slightly lowered uniformity was observed. Without wishing to be bound by theory, this reduction in uniformity stemmed from the reduction in the number of the A residues, namely in a reduction in the solubilizing characteristics of the oligomer, which has a direct effect on the film forming properties of the oligomer. The ×50 image of the film formed from the BBABBA oligomer suggests that the adhesion to the substrate is reduced. The ×100 image of the same film suggests that small microcrystalline domains may be forming in this film. These domains are well above 1 μm in size.

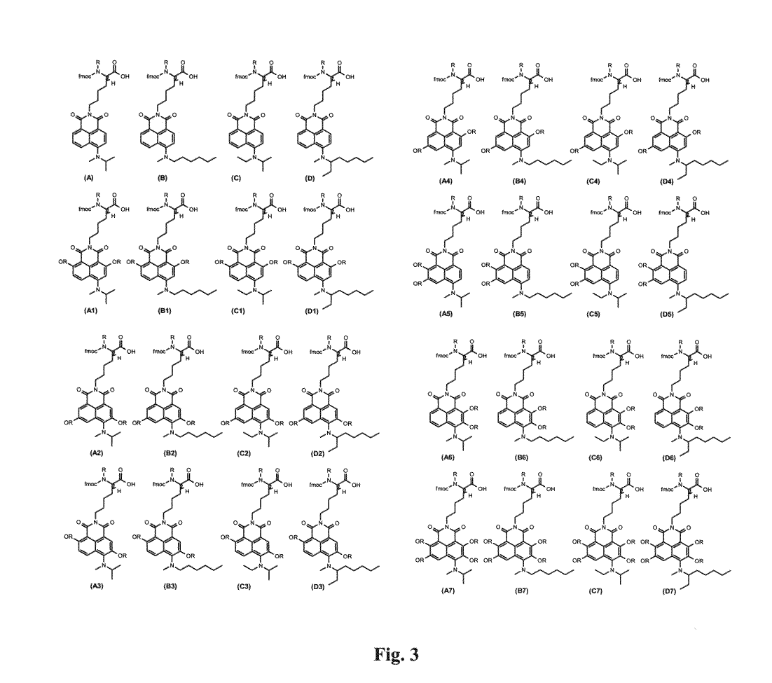

To test the intermolecular interactions which may be present outside the microcrystalline domains in the film of BBABBA, in comparison with the film of BABABA, Atomic Force Microscopy (AFM) images of the two films were taken. FIG. 2 shows that the topography of the two films (outside of the microcrystalline domains shown in the BBABBA film of FIG. 1) is very similar (left picture for each film). However, the phase contrast (shown in the right image for each of the films) is 3 times larger for the BBABBA film as compared to the BABABA film. This difference may attest to the difference in packing or other forms of molecular interactions which exist between the two films.

The optical activity of the two films, formed from Compounds C and D was studied both in solution and as solid films. The emission spectrum and the quantum efficiency were similar between the two types of oligomers. The photoluminescence (PL) efficiency decreased upon film forming from 50% in dilute solutions to about 20% in the solid pristine film.

The effect of blending each of the oligomers into a host matrix, such as PVK (ploy vinyl carbazole) was studied as well. When Compound C was blended as 25% (by weight) in PVK its PL efficiency was measured at about 40%. This reduced efficiency in the solid state, as compared with the dilute state, was indicative of intermolecular interactions in the solid film which are responsible for the quenching of the PL effect.

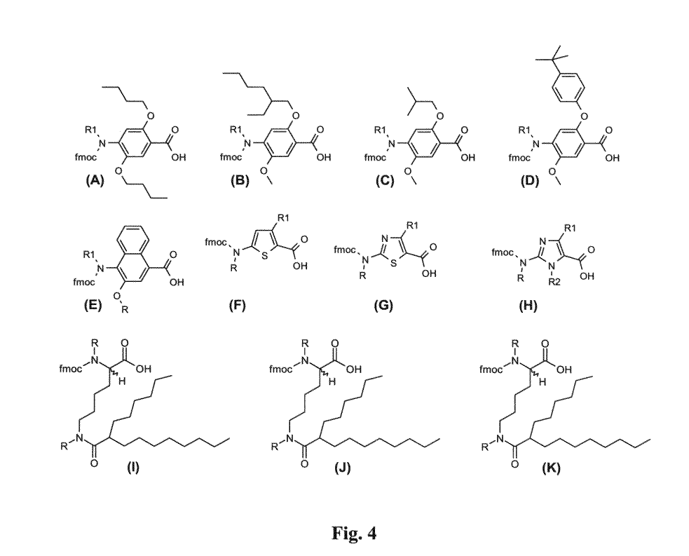

Different substitutions on any part of the compounds of general formulas VI to XI impart the resulting compounds with electronic and photoelectronic properties which otherwise may be unobtainable. By varying the luminescent groups, as shown in FIG. 3, one obtains highly versatile libraries of structurally similar materials with closely related properties. This allowed for the orthogonal optimization of luminescence properties such as luminescence spectra and yields and printability of the resulting materials.

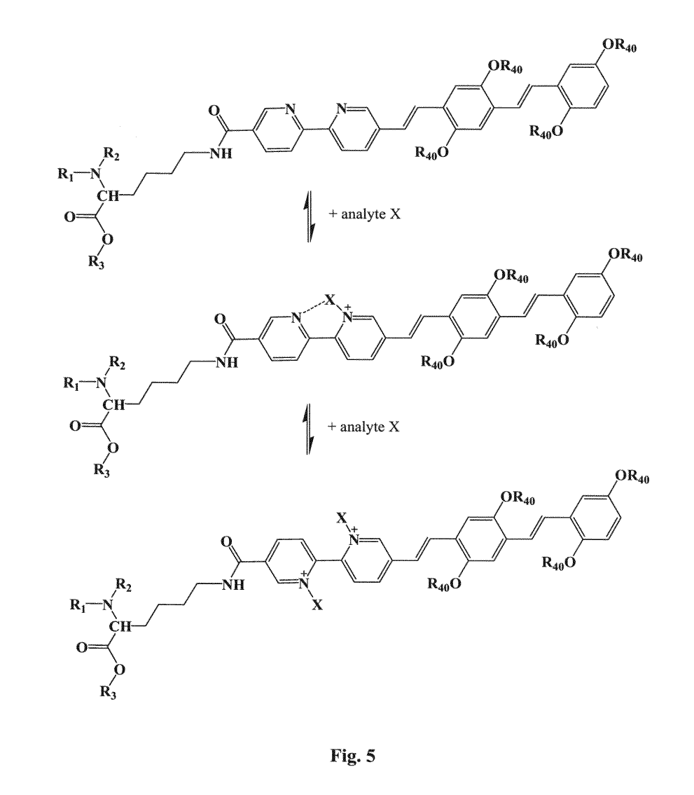

The material printing properties have also been tuned by using another group of compounds of the general formula I, which comprise π-conjugated amino acids and amino acids which bear different solubilizing moieties, as shown in FIG. 4. The optical properties of such compounds are dictated from the sequence of the π-conjugated acid monomers and their abundance. The material properties may also be tuned by varying the nature and sequence of side groups of the π-conjugated acids as well as by adding non-conjugated amino acids to the skeleton. The systems of FIG. 4 may also be varied by using different amino acid scaffolds as well as by using different enantiomers and/or diastereoisomers of the systems.

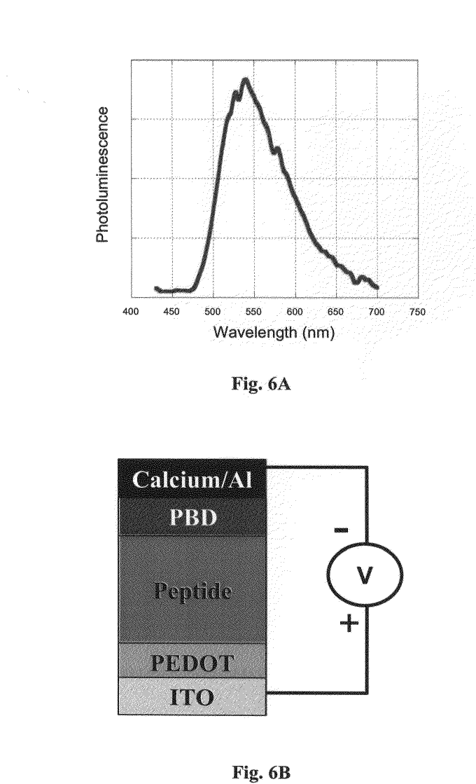

The compounds of the present invention may also be used as sensing molecules for the detection of various analytes such as protons in solution and alkylating agents in the liquid, solution, gas or solid states. For example, the compounds of any one of the general formulas XIII through XV may be used for the sensing of protons and alkylating groups. As shown in FIG. 5, upon bonding of an analyte molecule (e.g., H+ or an alkylating agent), the electronic and photoelectronic properties (e.g. photoluminescence, capacitance, or resistance) will vary as a result of the interruption in the conjugation which was present in the compound prior to analyte bonding.

The compounds of the present invention have also been used in the manufacturing of devices such as wires, resistors and emissive layers of light emitting diodes (LEDs). Films of oligomers were prepared by spin-coating at 2000 rpms from solutions of 30 mg oligomer in 1 ml CH₂Cl₂. For the PL efficiency measurements the oligomers were spin-coated on glass substrates and for the LEDs were spin-coated on ITO pre-coated by PEDOT (BAYTRON® P VP Al 4083).

The oligomers' PL efficiency was tested using the procedure described in J. C. deMello, H. F. Wittmann, and R. H. Friend, “An improved experimental determination of external photoluminescence quantum efficiency,” Adv. Mater., vol. 9, pp. 230, 1997. For a derivative of Compound E, having the following structure:

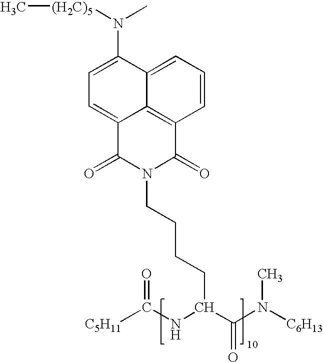

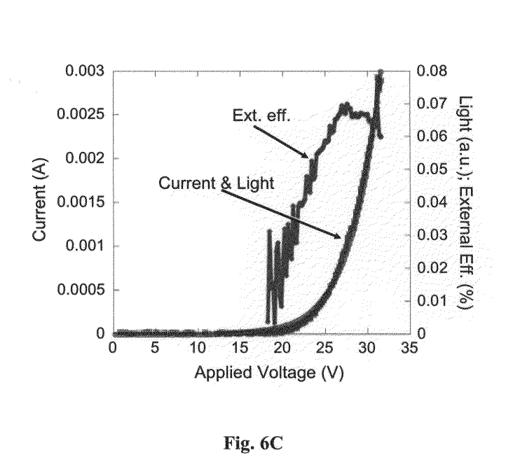

the PL efficiency was found to be ˜10% and the PL spectrum was centered at 540 nm as shown in FIG. 6A.

LEDs were prepared on glass/ITO substrates. The ITO was cleaned by solvents and oxygen plasma (conditions equivalent to etch of 350 nm of polyimide) prior to the deposition of the PEDOT layer. In order to avoid exposure to oxygen and humidity, all of the following steps were performed under inert conditions (<1 ppm O₂ and H₂O). The PEDOT was annealed at 110° C. under dry vacuum for 3 hr, before the oligomer film of the derivative of Compound E (shown above) was spin coated on top of it. The final film was annealed at 110° C. under dry vacuum for 3 hr. The oligomer film was next covered by a thin layer (˜30 nm) of sublimed 2-(4-biphenylyl)-5-phenyl-1,3,4-oxadiazole (PBD) which served as a hole and exciton blocking layer. Without breaking the vacuum, a top contact of 10 nm of Ca followed by 200 nm of Al was evaporated at 0.1 nm/sec and at a pressure of ˜5×10⁻⁷ atm. The schematic structure of the LED structure thus prepared is shown in FIG. 6B.

The LEDs were tested using a semiconductor parameter analyzer which applied voltage to the LED and measured the current flowing through it. It also simultaneously measured the voltage across a Si photodetector that collected the electroluminescence of the LED. The external quantum efficiency was calculated using the procedure described in N. C. Greenham, R. H. Friend, and D. D. C. Bradley, “Angular Dependence of the Emission From a Conjugated Polymer Light-Emitting Diode: Implications for Efficiency Calculations,” Adv. Mater., vol. 6, pp. 491-494, 1994. The LED exhibited a 0.07% as shown in FIG. 6C.

1-110. (canceled)

111. A method of making a printable material comprising a polymer whose units are amino acids or nucleic acids that are connected to each other by peptide bonds.

112. The method of claim 111, wherein the units are amino acids.

113. The method of claim 112, wherein the carboxy and amino groups of the amino acid is connected to Z, wherein Z is selected from the group consisting of C1-C2 alkylene, C5-C8 cycloalkylene, C5-C10 arylene and C5-C12 heteroarylene having at least one heteroatom selected from N, O, and S.

114. The method of claim 113, wherein Z is optionally attached to X, wherein X is selected from the group consisting of C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

115. The method of claim 114, wherein X is optionally attached to NR₄R₅, wherein R₄ and R₅, independently of each other, are selected from H, C1-C20 alkyl, C2-C20 alkenyl, C2-C20 alkynyl, C1-C20 alkylene, C2-C20 alkenylene, C2-C20 alkynylene, cycloalkyl, cycloalkenyl, cycloalkynyl, aryl, arylene, heteroaryl, heteroarylene, aralkyl, heteroaralkyl, haloalkyl, alkoxy, haloalkoxy, sulfonyl, carboxy, alkylaminocarbonyl and a radical of the general formula II: 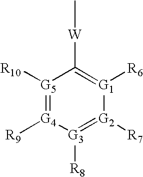
wherein each of R6 to R10, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl;
two vicinal (i.e., neighboring) groups of R6 to R10 may together with the carbon atoms to which they are bonded form a substituted or unsubstituted C5-C10 fused ring system selected from cycloalkyl, cycloalkenyl, cycloalkynyl, heterocyclic, and arylene; said fused ring system may contain at least one heteroatom selected from O, N or S;
wherein each of G1 to G5 may be an atom selected from C, O, N or S; where the atom G1, G2, G3, G4 or G5 is different from C, the atom may be charged or neutral; when atom G1 to G5 is different from C, the atom may or may not be substituted as shown; in case of substitution, said atom (G1 to G5) may be positively charged; when charged, the system may be accompanied by a counter ion selected from negatively inorganic or organic anions;
W is a group selected from —C(O)—, —S(O)— and —S(O)₂—;
or R4 and R5 together with the N atom to which they are bonded, may form a heterocyclic ring structure having optionally at least one additional heteroatom selected from N, O or S; said ring structure being selected from substituted or unsubstituted pyridine, isoquinoline, benzoisoquinoline, benzoisoquinoline-1-one, isobenzoquinoline-1,3-dione, benzo[1,7]naphthyridine dione, and 1,6,8-triazaphenalen-7,9-dione.

116. The method of claim 112, wherein said amino group is attached to R1, which is selected from the group consisting of H, C1-C20 alkyl, C2-C20 alkenyl, C2-C20 alkynyl, C1-C20 alkylene, C2-C20, alkenylene, C2-C20 alkynylene, silyl, C1-C20 alkylene carbonyl nucleobase and N-protecting group.

117. The method of claim 111, wherein said polymer is 2 to 6 units.

118. The method of claim 111, wherein said polymer is at least 10 units.

119. A method of making of varying at least one property of a printable material by including in said material a polymer whose units are amino acids or nucleic acids that are connected to each other by peptide bonds, wherein said property is selected from the group consisting of solubility, viscosity, film-forming, adhesivity, electronic, photoelectronic and magnetic.

120. The method of claim 119, wherein the units are amino acids.

121. The method of claim 120, wherein the carboxy and amino groups of the amino acid is connected to Z, wherein Z is selected from the group consisting of C1-C2 alkylene, C5-C8 cycloalkylene, C5-C10 arylene and C5-C12 heteroarylene having at least one heteroatom selected from N, O, and S.

122. The method of claim 121, wherein Z is optionally attached to X, wherein X is selected from the group consisting of C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl.

123. The method of claim 122, wherein X is optionally attached to NR₄R₅, R₄ and R₅, independently of each other, are selected from H, C1-C20 alkyl, C2-C20 alkenyl, C2-C20 alkynyl, C1-C20 alkylene, C2-C20 alkenylene, C2-C20 alkynylene, cycloalkyl, cycloalkenyl, cycloalkynyl, aryl, arylene, heteroaryl, heteroarylene, aralkyl, heteroaralkyl, haloalkyl, alkoxy, haloalkoxy, sulfonyl, carboxy, alkylaminocarbonyl, and a radical of the general formula II: 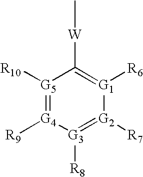
wherein each of R6 to R10, independently of each other, is selected from H, hydroxyl, amine, amide, nitro, halogen, C1-C20 alkyl, C2-C20, alkenyl, C2-C20 alkynyl, C1-C20 alkylene, C2-C20 alkenylene, C2-C20 alkynylene, C3-C6 cycloalkyl, cycloalkenyl or cycloalkynyl, aryl, arylene, C5-C15 heteroaryl, heteroarylene, aralkyl, heteroaralkyl, haloalkyl, alkoxy, haloalkoxy, sulfonyl, carboxy, and alkylaminocarbonyl;
two vicinal (i.e., neighboring) groups of R6 to R10 may together with the carbon atoms to which they are bonded form a substituted or unsubstituted C5-C10 fused ring system selected from cycloalkyl, cycloalkenyl, cycloalkynyl, heterocyclic, and arylene; said fused ring system may contain at least one heteroatom selected from O, N or S;
wherein each of G1 to G5 may be an atom selected from C, O, N or S; where the atom G1, G2, G3, G4 or G5 is different from C, the atom may be charged or neutral; when atom G1 to G5 is different from C, the atom may or may not be substituted as shown; in case of substitution, said atom (G1 to G5) may be positively charged; when charged, the system may be accompanied by a counter ion selected from negatively inorganic or organic anions;
W is a group selected from —C(O)—, —S(O)— and —S(O)₂—;
or R4 and R5 together with the N atom to which they are bonded, may form a heterocyclic ring structure having optionally at least one additional heteroatom selected from N, O or S; said ring structure being selected from substituted or unsubstituted pyridine, isoquinoline, benzoisoquinoline, benzoisoquinoline-1-one, isobenzoquinoline-1,3-dione, benzo[1,7]naphthyridine dione, and 1,6,8-triazaphenalen-7,9-dione.

124. The method of claim 120, wherein said amino group is attached to R1, which is selected from the group consisting of H, C1-C20 alkyl, C2-C20 alkenyl, C2-C20 alkynyl, C1-C20 alkylene, C2-C20, alkenylene, C2-C20 alkynylene, silyl, C1-C20 alkylene carbonyl nucleobase and N-protecting group.

125. The method of claim 119, wherein said polymer is 2 to 6 units.

126. The method of claim 119, wherein said polymer is at least 10 units.

127. The method of claim 119, wherein said property is solubility.

128. The method of claim 119, wherein said property is viscosity.

129. The method of claim 119, wherein said property is film-forming.
